SEQUENCE LISTING (4 sequences)

SEQ ID NO: 1 - protein, 329 aa, Homo sapiens
MVRTKTWTLKKHFVGYPTNSDFELKTAELPPLKNGEVLLEALFLTVDPYMRVAAKRLKEGDTMMGQQVAKVVESKNVALPKGTIVLASPGWTTHSISDGKDLEKLLTEWPDTIPLSLALGTVGMPGLTAYFGLLEICGVKGGETVMVNAAAGAVGSVVGQIAKLKGCKVVGAVGSDEKVAYLQKLGFDVVFNYKTVESLEETLKKASPDGYDCYFDNVGGEFSNTVIGQMKKFGRIAICGAISTYNRTGPLPPGPPPEIVIYQELRMEAFVVYRWQGDARQKALKDLLKWVLEGKIQYKEYIIEGFENMPAAFMGMLKGDNLGKTIVKA

SEQ ID NO: 2 - RNA, 1599 nt, Homo sapiens
  source 1..1599: mol_type = mRNA
gcagtcccagccgccttccccaggcagtggaaccttcgggctcctgagcttcaggatggttcgtactaagacatggaccctgaagaagcactttgttggctatcctactaatagtgactttgagttgaagacagctgagctcccacccttaaaaaatggagaggtcctgcttgaagctttgttcctcaccgtggatccctacatgagagtggcagccaaaagattgaaggaaggtgatacaatgatggggcagcaagtggccaaagttgtggaaagtaaaaatgtagccctaccaaaaggaactattgtactggcttctccaggctggacaacgcactccatttctgatgggaaagatctggaaaagctgctgacagagtggccagacacaataccactgtctttggctctggggacagttggcatgccaggcctgactgcctactttggcctacttgaaatctgtggtgtgaagggtggagaaacagtgatggttaatgcagcagctggagctgtgggctcagtcgtggggcagattgcaaagctcaagggctgcaaagttgttggagcagtagggtctgatgaaaaggttgcctaccttcaaaagcttggatttgatgtcgtctttaactacaagacggtagagtctttggaagaaaccttgaagaaagcgtctcctgatggttatgattgttattttgataatgtaggtggagagttttcaaacactgttatcggccagatgaagaaatttggaaggattgccatatgtggagccatctctacatataacagaaccggcccacttcccccaggcccacccccagagattgttatctatcaggagcttcgcatggaagcttttgtcgtctaccgctggcaaggagatgcccgccaaaaagctctgaaggacttgctgaaatgggtcttagagggtaaaatccagtacaaggaatatatcattgaaggatttgaaaacatgccagctgcatttatgggaatgctgaaaggagataatttggggaagacaatagtgaaagcatgaaaaagaggacacatggaatctggaggccatttagatgattagttaatttgtttttcaccatttagcaaaaatgtatactaccttaaatgtcttaagaaatagtactcataatgagtttgagctacttaataaaatacatttaagtggtatgtaattagtgatggaggatggaagtttcaaagtcaacaacaaccagtcacctcactgttcactacagcttcttctgagttgtggtagaaaataatggctttgaagtgtgaaaattttccaaacactgtatgttttcattcataagtgggagctgaacaatgagaacacatggacatagggaggggaacaacacacgctggggcctgtggaagccggggtgagggagggagagcatcaggataaatagctaatgccttcgggggctaatacctaggtgatgggttgacaggtgaagcaaaccatcatggcacacgtttacctatgtaacaaacctgcacattccacacaattatcccggaactgaaaataaaataaaattaaattaaaaaaaaaagttagccggg

SEQ ID NO: 3 - protein, 301 aa, Homo sapiens
MVRTKTWTLKKHFVGYPTNSDFELKTAELPPLKNGEVLLEALFLTVDPYMRVAAKRLKEGDTMMGQQVAKVVESKNVALPKGTIVLASPGWTTHSISDGKDLEKLLTEWPDTIPLSLALGTVGMPGLTAYFGLLEICGVKGGETVMVNAAAGAVGSVVGQIAKLKGCKVVGAVGSDEKVAYLQKLGFDVVFNYKTVESLEETLKKASPDGYDCYFDNVGGEFSNTVIGQMKKFGRIAICGAISTYNRTGPLPPGPPPEIVIYQELRMEAFVVYRWQGDARQKALKDLLKWVLEIKRENEED

SEQ ID NO: 4 - RNA, 1012 nt, Homo sapiens
  source 1..1012: mol_type = mRNA
gcagtcccagccgccttccccaggcagtggaaccttcgggctcctgagcttcaggatggttcgtactaagacatggaccctgaagaagcactttgttggctatcctactaatagtgactttgagttgaagacagctgagctcccacccttaaaaaatggagaggtcctgcttgaagctttgttcctcaccgtggatccctacatgagagtggcagccaaaagattgaaggaaggtgatacaatgatggggcagcaagtggccaaagttgtggaaagtaaaaatgtagccctaccaaaaggaactattgtactggcttctccaggctggacaacgcactccatttctgatgggaaagatctggaaaagctgctgacagagtggccagacacaataccactgtctttggctctggggacagttggcatgccaggcctgactgcctactttggcctacttgaaatctgtggtgtgaagggtggagaaacagtgatggttaatgcagcagctggagctgtgggctcagtcgtggggcagattgcaaagctcaagggctgcaaagttgttggagcagtagggtctgatgaaaaggttgcctaccttcaaaagcttggatttgatgtcgtctttaactacaagacggtagagtctttggaagaaaccttgaagaaagcgtctcctgatggttatgattgttattttgataatgtaggtggagagttttcaaacactgttatcggccagatgaagaaatttggaaggattgccatatgtggagccatctctacatataacagaaccggcccacttcccccaggcccacccccagagattgttatctatcaggagcttcgcatggaagcttttgtcgtctaccgctggcaaggagatgcccgccaaaaagctctgaaggacttgctgaaatgggtcttagagatcaaaagagaaaatgaagaagattgaagcttcaaagcagaaaaatgaagggaatatgtatcattcaccattacctta

(19) United States
(12) Patent Application Publication
(10) Pub. No.: US 2024/0325328 A1
(43) Pub. Date: Oct. 3, 2024

(54) TREATMENT OF BRAIN METASTASES AND CNS METASTASES USING ILLUDINS OR HYDROXYLUREAMETHYL ACYLFULVENE

(71) Applicant: Lantern Pharma Inc., Dallas, TX (US)

(72) Inventors: Kishor BHATIA, Dallas, TX (US); Aditya KULKARNI, Dallas, TX (US)

(21) Appl. No.: 18/349,256

(22) Filed: Jul. 10, 2023

Related U.S. Application Data
(63) Continuation of application No. PCT/US2022/070126, filed on Jan. 10, 2022.
(60) Provisional application No. 63/135,370, filed on Jan. 8, 2021.

(51) Int. Cl. A61K 31/17; A61K 45/06; C12Q 1/6886; G01N 33/68

(52) U.S. Cl. CPC A61K 31/17; A61K 45/06; C12Q 1/6886; G01N 33/68; C12Q 2600/106; C12Q 2600/112; C12Q 2600/158; G01N 2333/90206; G01N 2800/52

(57) ABSTRACT

This application discloses methods for treating a cancer that has metastasized to the brain in a subject using a therapeutically effective amount of HydroxyUreaMethyl Acylfulvene. In addition, pharmaceutical compositions having HydroxyUreaMethyl Acylfulvene and a pharmaceutically acceptably carrier, diluent, excipient, or a combination thereof are disclosed.

### CROSS-REFERENCE

This application is a continuation of U.S. Patent Application No. PCT/US2022/070126, filed Jan. 10, 2022, which claims the benefit of U.S. Provisional Application No. 63/135,370, filed Jan. 8, 2021, the entirety of which is incorporated herein by reference.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### INCORPORATION BY REFERENCE OF SEQUENCE LISTING

The Sequence Listing titled 197585010188.XML, which was created on May 6, 2024, and is 8 kilobytes in size, is hereby incorporated by reference in its entirety.

### TECHNICAL FIELD

This application relates to the field of chemistry and oncology. More particularly, this application relates to methods for treating brain metastases using illudins and Hydroxy UreaMethyl Acylfulvene. The present invention is directed to methods of treating CNS metastasis.

### BACKGROUND

The majority of brain metastasis or metastases cancer patients generally has a poor outcome. Central nervous (CNS) metastasis, notably in the brain, are prevalent in lung cancer (20-56% of patients), breast cancer (5-20% of patients) and melanoma (7-16% of patients). The incidence of brain metastases is thought to be increasing, owing to improved systemic therapy that fails to control the disease in the brain. The development of brain metastases impairs patient survival and is the cause of death in up to 50% of patients. Hallmarks of brain metastasis development have been identified.

Therapeutically intervening in brain metastases has been a particular challenge for oncologists since metastatic tumors are generally resistant to many chemotherapy agents and surgical resection options that preserve brain function are limited. The brain's unique microenvironment poses a formidable barrier to metastatic cancer cells and treatments. The brain metastatic tumor cell must traverse the blood brain barrier (BBB). The BBB is heterogeneously and impermeable to most drugs. Until recently, brain metastasis therapy was primarily local including surgery, stereotactic radiation therapy, and whole brain radiation therapy. Currently treatment options are limited.

Accordingly, there is always a need for therapeutic interventions to treat brain metastases.

### SUMMARY

Provided herein are methods for treating, suppressing or decreasing brain/CNS metastasis or metastases or metastatic brain tumors in a subject having a cancer, comprising administering to the subject a therapeutically effective amount of illudins or Hydroxy UreaMethyl Acylfulvene, particularly the optical isomer having a negative optical activity. The cancer can be selected from the group consisting of lung cancer, breast cancer, melanoma, colon cancer, kidney cancer, renal cell carcinoma, mesothelioma, ovarian cancer, pancreatic cancer, sarcoma, leukemia, lymphoma, urothelial cancer, head and neck cancer, osteosarcoma, glioblastoma, astrocytoma, and bladder cancer. Further provided herein are methods for treating or decreasing brain metastasis in a subject having a cancer, wherein the illudin(s) or Hydroxy UreaMethyl Acylfulvene is administered as a monotherapy. Further provided herein are methods for treating or decreasing brain metastasis in a subject having a cancer, wherein the Hydroxy UreaMethyl Acylfulvene is administered as a combination therapy, wherein the combination therapy comprises administration of Hydroxy UreaMethyl Acylfulvene and at least one therapeutic agent selected from the group consisting of temozolomide, bevacizumab, everolimus, carmustine, lomustine, procarbazine, vincristine, irinotecan, cisplatin, carboplatin, paclitaxel, methotrexate, etoposide, vinblastine, bleomycin, actinomycin, cyclophosphamide, and ifosfamide. The methods for treating or decreasing brain metastasis in a subject having a cancer may further comprise subjecting the subject to a radiation therapy. The radiation therapy is selected from the group consisting of whole-brain irradiation, fractionated radiotherapy, radiosurgery, and a combination thereof.

Provided herein are methods for treating or decreasing brain or CNS metastasis in a subject, wherein administering an effective amount of Hydroxy UreaMethyl Acylfulvene to a subject in need thereof results in an inhibition of brain metastasis in the subject by more than 10%. 20%. 30%, 40%, 50%, 60%, 70%. 80%, 90%, or 100% relative to brain metastasis not exposed to the treatment.

Provided herein are methods for treating or decreasing brain metastasis in a subject having a cancer, wherein the cancer is associated with an increased expression of prostaglandin reductase 1 (PTGR1) enzyme. Further provided herein are methods for treating or decreasing brain metastasis in a subject having a cancer, wherein the cancer is associated with an increased expression of the nucleic acid sequence as set forth in SEQ ID NO:2 or SEQ ID NO:4. Further provided herein are methods for treating or decreasing brain metastasis in a subject having a cancer, wherein the cancer is associated with an increased expression of the amino acid sequence as set forth in SEQ ID NO:2 or SEQ ID NO:4.

Provided herein are methods for treating or decreasing brain metastasis in a subject having a cancer comprising: (a) determining expression of PTGR1 gene in a sample obtained from the subject: (b) selecting the subject having an increased expression level of PTGR1 gene in step a; and (c) administering to the subject selected in step b an effective amount of Hydroxy UreaMethyl Acylfulvene, thereby treating or decreasing brain metastasis in the subject. Further provided herein are methods for treating or decreasing brain metastasis in a subject having a cancer comprising: (a) determining expression of PTGR1 protein in a sample obtained from the subject: (b) selecting the subject having an increased expression level of PTGR1 protein in step a; and (c) administering to the subject selected in step b an effective amount of Hydroxy UreaMethyl Acylfulvene, thereby treating or decreasing brain metastasis in the subject. Further provided herein are methods for treating or decreasing brain metastasis in a subject having a cancer is sufficient to metabolize Hydroxy UreaMethyl Acylfulvene.

Provided herein are methods for treating or decreasing brain metastasis in a subject having a cancer, comprising administering to the subject a therapeutically effective amount of: (a) a first pharmaceutical composition, wherein the first pharmaceutical composition comprises a therapeutically effective amount of Hydroxy UreaMethyl Acylfulvene and a pharmaceutically acceptable carrier, and (b) a second pharmaceutical composition, wherein the second pharmaceutical composition comprises an additional therapeutic agent and a pharmaceutically acceptable carrier. The first pharmaceutical composition and the second pharmaceutical composition are administered as one composition or as separate compositions. The additional therapeutic agent is selected from the group consisting of temozolomide, bevacizumab, everolimus, carmustine, lomustine, procarbazine, vincristine, irinotecan, cisplatin, carboplatin, paclitaxel, methotrexate, etoposide, vinblastine, bleomycin, actinomycin, cyclophosphamide, and ifosfamide.

Provided herein are methods for treating or decreasing brain metastasis in a subject having a cancer comprising: (a) determining expression of PTGR1 gene in a sample obtained from the subject: (b) selecting the subject having an increased expression level of PTGR1 gene in step a; and (c) administering to the subject selected in step b an effective amount of Hydroxy UreaMethyl Acylfulvene, thereby treating or decreasing brain metastasis in the subject, wherein the cancer can be selected from the group consisting of lung cancer, breast cancer, melanoma, colon cancer, kidney cancer, renal cell carcinoma, mesothelioma, ovarian cancer, pancreatic cancer, sarcoma, leukemia, lymphoma, urothelial cancer, head and neck cancer, osteosarcoma, glioblastoma, astrocytoma, and bladder cancer.

Provided herein are methods of inhibiting brain metastasis, comprising contacting the brain metastasis with Hydroxy UreaMethyl Acylfulvene, wherein the contacting comprises administering a therapeutically effective amount of Hydroxy UreaMethyl Acylfulvene to a subject having the brain metastasis. Provided herein are methods of inhibiting brain metastasis, comprising contacting the brain metastasis with Hydroxy UreaMethyl Acylfulvene, wherein the brain metastasis is associated with increased expression of prostaglandin reductase 1 (PTGR1) enzyme.

Provided herein are methods of inducing apoptosis in brain metastasis, comprising contacting a brain tumor cell with Hydroxy UreaMethyl Acylfulvene, wherein the contacting comprises administering an effective amount of Hydroxy UreaMethyl Acylfulvene to a subject having the brain metastasis.

Provided herein are methods of inhibiting or decreasing brain metastasis in a subject having a cancer, comprising: (a) measuring the level of expression of PTGR1 gene or the protein for which it encodes in a biological sample obtained from the subject. (b) comparing the level of expression of PTGR1 gene or the protein for which it encodes in said sample against a standard of expression of PTGR1 gene or the protein for which it encodes, and (c) where the level of expression of PTGR1 gene or the protein for which it encodes is increased or high, administering a therapeutically effective amount of Hydroxy UreaMethyl Acylfulvene thereby inhibiting or decreasing the brain metastasis.

Provided herein are methods of inhibiting or decreasing brain metastasis in a subject having a cancer, comprising: (a) measuring the level of expression of PTGR1 gene or the protein for which it encodes in a biological sample obtained from the subject, (b) comparing the level of expression of PTGR1 gene or the protein for which it encodes in said sample against a standard of expression of PTGR1 gene or the protein for which it encodes, and (c) where the level of expression of PTGR1 gene or the protein for which it encodes is at or above a threshold for Hydroxy UreaMethyl Acylfulvene sensitivity, administering a therapeutically effective amount of HydroxyUreaMethyl Acylfulvene thereby inhibiting or decreasing the brain metastasis. Further provided herein are methods of inhibiting or decreasing brain metastasis in a subject having a cancer, wherein determining the Hydroxy UreaMethyl Acylfulvene sensitivity comprises using a 3D model of PDX-derived brain metastasis.

Provided herein are kits for use in determining sensitivity of a specimen to Hydroxy UreaMethyl Acylfulvene according to the method of any one of claim 1-36, wherein the kit comprises one or more reagents, standards, and instructions for use thereof, wherein the standards comprise expression or transcription of PTGR1, providing a threshold level, or a target level for screening sensitivity of the specimen to the Hydroxy UreaMethyl Acylfulvene.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the apparent permeability of LP-184 and Temozolomide (TMZ).

FIG. 2 shows the impact of LP-184 on cell viability and BBB integrity.

FIG. 3 shows the analysis of GEO dataset, GSE100534.

FIG. 4 shows the analysis of GEO, dataset GSE132226.

FIG. 5 shows the potency of LP-184 in 3D models of PDX-derived brain metastases.

### DEFINITIONS

Unless defined otherwise, all technical and scientific terms used herein have the same meaning as is commonly understood by one of skill in art to which the subject matter herein belongs. As used herein, the following definitions are supplied in order to facilitate the understanding of this application.

As used herein, the terms a “patient,” “subject,” “individual,” and “host” refer to either a human or a non-human animal suffering from or suspected of suffering from a disease or disorder associated with aberrant biological or cell growth activity or a brain metastasis.

The terms “treat” and “treating” such a disease or disorder refers to ameliorating at least one symptom of the disease or disorder. These terms, when used in connection with a condition such as cancer, refer to one or more of: impeding growth of the cancer, causing the cancer to shrink by weight or volume, extending the expected survival time of the patient, inhibiting tumor growth, reducing tumor mass, reducing size or number of metastatic lesions, inhibiting the development of new metastatic lesions, prolonging survival, prolonging progression-free survival, prolonging time to progression, and/or enhancing quality of life. The terms “treatment” or “treating” a brain metastasis can include arresting the development or reversing the symptom or symptoms of a brain metastasis and/or an improvement in clinical outcome of the patient suffering from a brain metastasis. Example of improvements in clinical outcome include longer survival time, reduction in tumor size, non-growth in tumor size, and/or lack of exacerbation in neurological symptoms.

The term “preventing” when used in relation to a condition or disease such as cancer, refers to a reduction in the frequency of, or delay in the onset of, symptoms of the condition or disease. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

The term “pharmaceutically acceptable” means that, which is useful in preparing a pharmaceutical composition that is generally safe, non-toxic, and neither biologically nor otherwise undesirable and includes that which is acceptable for veterinary as well as human pharmaceutical use.

The term “therapeutic effect” refers to a beneficial local or systemic effect in animals, particularly mammals, and more particularly humans, caused by administration of a compound or composition of the invention. The phrase “therapeutically-effective amount” means that amount of a compound or composition of the invention that is effective to treat a disease or condition caused by a brain metastasis at a reasonable benefit/risk ratio. In some embodiments, the therapeutically effective amount of HydroxyUreaMethyl Acylfulvene or a pharmaceutically acceptable salt thereof is selected from the group consisting of 1 mg/day, 2 mg/day, 4 mg/day, 5 mg/day, 10 mg/day, 15 mg/day, 20 mg/day, 30 mg/day, 60 mg/day, 90 mg/day, 120 mg/day, 150 mg/day, 180 mg/day, 210 mg/day, 240 mg/day, 270 mg/day. 300 mg/day, 360 mg/day, 400 mg/day, 440 mg/day, 480 mg/day, 520 mg/day 580 mg/day, 600 mg/day, 620 mg/day, 640 mg/day, 680 mg/day, and 720 mg/day.

The therapeutically effective amount of such substance will vary depending upon the subject and disease condition being treated, the weight and age of the subject, the severity of the disease condition, the manner of administration and the like, which can readily be determined by one of skill in the art.

Prostaglandin reductase-1 (Ptgr1) is an alkenal/one oxidoreductase that is involved in the catabolism of eicosanoids and lipid peroxidation such as 4-hydroxynonenal (4-HNE). The protein and mRNA sequences of PTGR1 isoform 1 are set forth in SEQ ID NO: 1 and SEQ ID NO: 2, respectively. The protein and mRNA sequences of PTGR1 isoform 2 are set forth in SEQ ID NO:3 and SEQ ID NO:4, respectively.

The term “expression level” as used herein may refer to protein, RNA, or mRNA level of a particular gene of interest (for example, PTGR1). Any methods as described herein and/or known in the art can be utilized to determine the expression level. Examples include, but are not limited to, reverse transcription and amplification assays (such as PCR, ligation RT-PCR or quantitative RT-PCT), hybridization assays, Northern blotting, dot blotting, in situ hybridization, gel electrophoresis, capillary electrophoresis, column chromatography, Western blotting, immunohistochemistry, immunostaining, or mass spectrometry. Assays can be performed directly on biological samples or on protein/nucleic acids isolated from the samples. It is routine practice in the relevant art to carry out these assays. For example, the measuring step in any method described herein includes contacting the nucleic acid sample from the biological sample obtained from the subject with one or more primers that specifically hybridize to the gene of interest presented herein. Alternatively, the measuring step of any method described herein includes contacting the protein sample from the biological sample obtained from the subject with one or more antibodies that bind to the biomarker of the interest presented herein.

An increased expression level of PTGR1 gene can include an increase in its expression level by at least 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 1000%, 1500%, or more compared to a reference value or the expression level of this gene measured in a different (or previous) sample obtained from the same subject.

A “reference or baseline level/value” as used herein can be used interchangeably and is meant to be relative to a number or value derived from population studies, including without limitation, such subjects having similar age range, disease status (e.g., stage), subjects in the same or similar ethnic group, or relative to the starting sample of a subject undergoing treatment for cancer. Such reference values can be derived from statistical analyses and/or risk prediction data of populations obtained from mathematical algorithms and computed indices of cancer. Reference indices can also be constructed and used using algorithms and other methods of statistical and structural classification.

In some embodiments of the present invention, the reference or baseline value is the expression level of PTGR1 gene in a control sample derived from one or more healthy subjects or subjects who have not been diagnosed with any cancer.

In some embodiments of the present invention, the reference or baseline value is the expression level of PTGR1 gene in a sample obtained from the same subject prior to any cancer treatment. In other embodiments of the present invention, the reference or baseline value is the expression level of PTGR1 gene in a sample obtained from the same subject during a cancer treatment. Alternatively, the reference or baseline value is a prior measurement of the expression level of PTGR1 gene in a previously obtained sample from the same subject or from a subject having similar age range, disease status (e.g., stage) to the tested subject.

As used herein, the phrase “brain metastasis associated with cells” in which the functional activity of PTGR1 is high refers to a cancer comprising cells in which the functional activity of PTGR1 is likely to be high, or where the functionally activity of SMARCB1 in those cells have been validated to be high.

The term “sample” as used herein, refer to any biological sample derived from the subject, includes but is not limited to, cells, tissues samples, body fluids (including, but not limited to, mucus, blood, plasma, serum, urine, saliva, and semen), tumor cells, and tumor tissues. Samples can be provided by the subject under treatment or testing. Alternatively, samples can be obtained by the physician according to routine practice in the art.

The term “sensitivity.” “responsive” and “responsiveness,” as used herein, refer to the likelihood that a cancer treatment (e.g., LP184) has (e.g., induces) a desired effect, or, alternatively, refer to the strength of a desired effect caused or induced by the treatment in a cell (e.g., a cancer cell), a tissue (e.g., a tumor), or a patient having cancer (e.g., a human having cancer). For example, the desired effect can include inhibition of the growth of a cancer cell in vitro by more than 10%, 20%, 30%, 40%, 50%, 60%, 70%, 80%, 90%, or 100% relative to the growth of a cancer cell not exposed to the treatment. The desired effect can also include reduction in brain metastasis by, e.g., about 10%, 20%, 30%, 40%, 50%, 60%, 70%, 80%, 90%, or 100%. Sensitivity to treatment may be determined by a cell proliferation assay, e.g., a cell-based assay, which measures the growth of treated cells as a function of the absorbance of the cells of an incident light beam, such as the NCI60 assays described herein. In this assay, lesser absorbance indicates lesser cell growth, and thus, sensitivity to the treatment. A greater reduction in growth indicates more sensitivity to the treatment.

As used herein, the terms “treatment,” “treating,” and the like, refer to administering an agent, or carrying out a procedure, for the purposes of obtaining an effect. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of a tumor in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease: (b) inhibiting the disease, i.e., arresting its development; and (c) relieving the disease, i.e., causing regression of the disease.

### DETAILED DESCRIPTION

The application discloses or provides therapeutic methods for treating a brain metastasis using a Hydroxy UreaMethyl Acylfulvene (currently, termed as LP-184 by Lantern Pharma, Inc.), which can be a semisynthetic or synthetic antitumor agent derived from the mushroom toxin illudin S. Hydroxy UreaMethyl Acylfulvene is shown below and Hydroxy UreaMethyl Acylfulvene with negative optical activity was more effective in such treatments.

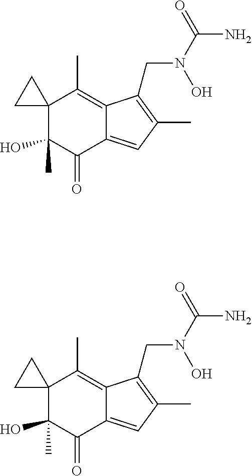 [SMILES: CC1=C(CN(O)C(N)=O)C2=C(C)C3(CC3)[C@@](C)(O)C(=O)C2=C1 ; CC1=C(CN(O)C(N)=O)C2=C(C)C3(CC3)[C@](C)(O)C(=O)C2=C1]

In certain embodiments, this application provides methods for treating brain metastasis. “Metastasis,” as used herein, refers to the presence of one or more cancer cells at a location that is not physically contiguous with the original location of the cancer (e.g., primary cancer). For example, and not by way of limitation, the cancer can include lung cancer, breast cancer, melanoma, colon cancer, kidney cancer, renal cell carcinoma, mesothelioma, ovarian cancer, pancreatic cancer, sarcoma, leukemia, lymphoma, urothelial cancer, head and neck cancer, osteosarcoma and bladder cancer. In certain embodiments, the cancer can include glioblastoma and astrocytoma. CNS or brain metastasis can be diagnosed by a clinician.

Specific embodiments relate to methods of treating brain metastases. The methods include the administration of an effective amount of HydroxyUreaMethyl Acylfulvene, particularly the optical isomer having a negative optical activity to a subject in need thereof. Patients or subjects suffering from a brain metastasis can be those arising from a melanoma, lung cancer, breast cancer, colon cancer, or kidney cancer. The therapeutic method generally entails administering a dose of a therapeutically effective amount of a formulation comprising Hydroxy UreaMethyl Acylfulvene to the patient. In one example, Hydroxy UreaMethyl Acylfulvene can be administered as a monotherapy. In other examples, Hydroxy UreaMethyl Acylfulvene can be administered as a combination therapy. HydroxyUreaMethyl Acylfulvene with negative optical activity was effective in such treatments.

One embodiment includes co-administering Hydroxy UreaMethyl Acylfulvene and an additional therapeutic agent in separate compositions or the same composition. Thus, some embodiments include a first pharmaceutical composition comprising: (a) a therapeutically effective amount of HydroxyUreaMethyl Acylfulvene or pharmaceutically acceptable salts thereof and (b) a pharmaceutically acceptable carrier, diluent, excipient or combination thereof: and a second pharmaceutical composition comprising: (a) a therapeutically effective amount of an additional therapeutic agent and (b) a pharmaceutically acceptable carrier, diluent, excipient or combination thereof. Some embodiments include a pharmaceutical composition comprising: (a) a therapeutically effective amount of an additional therapeutic agent; and (b) a pharmaceutically acceptable carrier, diluent, excipient or combination thereof. In some embodiments, the method described herein can further include subjecting the subject to a radiation therapy. In some embodiments, the radiation therapy can be whole-brain irradiation, fractionated radiotherapy, and radiosurgery. The subject should be diagnosed as having CNS and/or brain metastasis.

In some embodiments, the additional therapeutic agent is selected from the group consisting of temozolomide, bevacizumab, everolimus, carmustine, lomustine, procarbazine, vincristine, irinotecan, cisplatin, carboplatin, paclitaxel, methotrexate, etoposide, vinblastine, bleomycin, actinomycin, cyclophosphamide, and ifosfamide.

Another embodiment includes a method of inhibiting or decreasing brain metastasis in a subject having a cancer. The steps include determining medically whether the subject has the brain metastasis: measuring the level of expression of PTGR1 gene or the protein for which it encodes in a biological sample obtained from the subject, and comparing the level of expression of PTGR1 gene or the protein for which it encodes in said sample against a standard of expression of PTGR1 gene or the protein for which it encodes. The level of expression of PTGR1 gene or the protein for which it encodes may be increased or high and in which case, the method includes administering a therapeutically effective amount of Hydroxy UreaMethyl Acylfulvene thereby inhibiting, treating, suppressing, or decreasing the brain metastasis.

Brain metastases can be usually detected with an imaging test, typically computed tomography (CT) scans and magnetic resonance imaging (MRI) scans. Positron emission tomography (PET) scans, which use a radioactive substance called a tracer to search for disease, can also detect tumors when they become large, but these scans are much less sensitive and should not be relied upon for looking for brain metastases. If a tumor is found in the brain on CT or MRI and there is no pre-existing diagnosis of cancer, the doctors will typically get scans of the rest of the body to determine if the cancer came from outside the brain. If a source is found in the body, then a biopsy can be obtained from there rather than from the brain, and the brain tumor can be presumed to be related to the cancer found in the body. If the only tumor found is the one in the brain, a biopsy in the brain may be required to determine whether it is cancer and, if so, where it originated.

Some embodiments relate to a method of inhibiting a brain metastasis, the method including contacting the brain metastasis with Hydroxy UreaMethyl Acylfulvene. In some embodiments, the contacting comprises administering an effective amount of Hydroxy UreaMethyl Acylfulvene to a subject. In some embodiments, the method can be used to treat primary CNS tumors or a brain metastasis.

One embodiment includes a method for treating central nervous system (CNS) metastasis in a subject, comprising: a) determining whether a subject has CNS metastasis diagnosing the subject as having CNS metastasis and b) administering to the subject diagnosed as having CNS metastasis a therapeutically effective amount of Hydroxy UreaMethyl Acylfulvene thereby inhibiting, treating, suppressing, or decreasing the CNS metastasis.

Some embodiments relate to a method of inducing apoptosis in a brain metastasis, the method including contacting the brain tumor cell with Hydroxy UreaMethyl Acylfulvene. In some embodiments, the contacting comprises administering an effective amount of Hydroxy UreaMethyl Acylfulvene to a subject having the brain metastasis.

The administration period can be a multi-week treatment cycle as long as the tumor remains under control and the regimen is clinically tolerated. In some embodiments, a single dosage of Hydroxy UreaMethyl Acylfulvene or other therapeutic agent can be administered once a week, and preferably once on each of day 1 and day 8 of a three-week (21 day) treatment cycle. In some embodiments, a single dosage of Hydroxy UreaMethyl Acylfulvene or other therapeutic agent can be administered once a week, twice a week, three times per week, four times per week, five times per week, six times per week, or daily during a one-week, two-week, three-week, four-week, or five-week treatment cycle (or more). The administration can be on the same or different day of each week in the treatment cycle.

Hydroxy UreaMethyl Acylfulvene can be mainly administered by parenteral administration, specifically including subcutaneous administration, intramuscular administration, intravenous administration, transcutaneous administration, intrathecal administration, epidural administration, intra joint administration and local administration, or may also be administered in various dosage forms, for example by oral administration if possible.

The injections for parenteral administration include for example sterile, aqueous or non-aqueous solutions, suspensions and emulsions. The aqueous solutions and suspensions include for example distilled water for injections and physiological saline. The non-aqueous solutions and suspensions include for example propylene glycol, polyethylene glycol, vegetable oils such as olive oil, alcohols such as ethanol, and Polysorbate 80 (under trade name). Such composition may contain auxiliary agents such as preservatives, moistening agents, emulsifying agents, dispersing agents, stabilizers (for example, lactose) and dissolution auxiliary agents (for example, meglumine). These are sterilized by filtering through bacteria-retaining filters, blending sterilizing agents, or irradiation. Alternatively, these may be produced once into a sterile solid composition and then dissolved or suspended in sterile water or sterile solvents for injections, prior to use.

In some embodiments, the brain tumor can be a metastatic brain tumor, anaplastic astrocytoma, glioblastoma multiforme, oligodendroglioma, ependymomas, meningioma, mixed glioma, and a combination thereof. In some embodiments, the brain tumor is a glioblastoma multiforme. In some embodiments, the brain tumor is a metastatic brain tumor.

The liquid composition for oral administration includes for example pharmaceutically acceptable emulsions, liquids, suspensions, syrups, and elixirs and contains inert diluents for general use, for example distilled water and ethanol. The composition may contain auxiliary agents such as moistening agents and suspending agents, sweetening agents, flavoring agents, aromatic agents, and preservatives, other than the inert diluents.

It should also be understood that a specific dosage and treatment regimen for any patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the compound in the composition.

### EXAMPLES

Hydroxy UreaMethyl Acylfulvene having a negative chirality or LP-184 permeates the blood brain barrier in vitro 3D model, which closely recapitulates the human blood brain barrier. This model mimics transport properties of the BBB due to the formation of tight junctions, higher expression of specific carriers, and/or great cell viability. This 3D in vitro model of the BBB created by co-culturing brain endothelial cells, with pericytes and astrocytes layered in an insert, improves endothelial cell polarization, and enhances the formation of tight junctions, provides better endothelial cell-to-cell contact that is important for barrier development, and prevents the dilution of secreted neurotrophic factors. These conditions collectively lead to the development of an in vitro model that mimics the BBB.

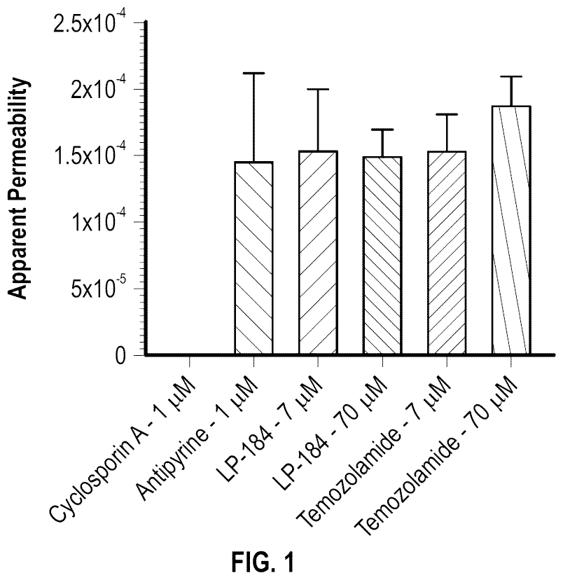

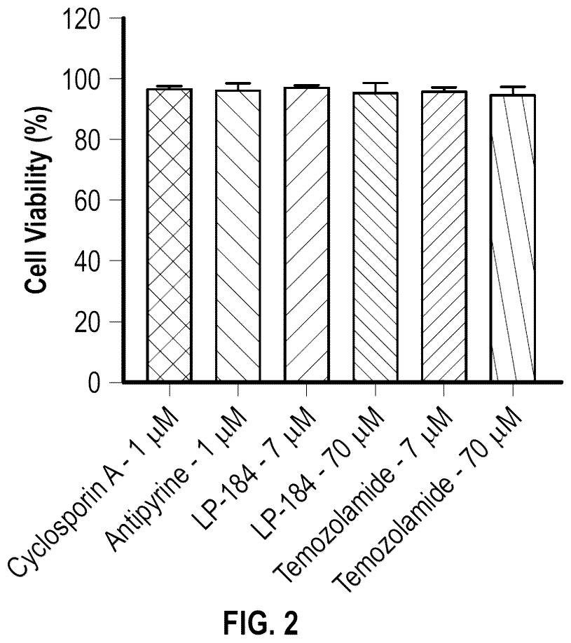

This assay leverages the novel 3D BBB model that allows studying both compound transport across the barrier as well as the effect of compounds on the structure and function of the BBB. Along with LP-184 and Temozolomide (TMZ), the standard of care agent for brain tumors, the example shows a comparison of the behavior of known positive and negative control agents Antipyrine and Cyclosporin A, respectively, as benchmarks. LP-184 was as effective as the standard of care drug TMZ in penetrating the blood brain barrier. Apparent BBB permeability measured for TMZ was 1.72*10⁻⁴ cm/s at 30 minutes and for LP-184 was 1.53*10⁻⁴ cm/s at 30 minutes (FIG. 1). Upon analyzing the modulation of the BBB due to compound interaction and treatment, it was observed that LP-184 had insignificant impact on cell viability and BBB integrity was not compromised (FIG. 2).

Clinical data analyses shows that PTGR1 levels in tumors metastasized to the brain are sufficient to allow LP-184 to be metabolized. Two Gene Expression Omnibus (GEO) datasets, GSE100534 and GSE132226, were obtained from National Center of Biotechnology Information (NCBI) to determine the threshold of PTGR1 levels in brain metastatic tissue for LP-184.

As shown in Table 1 below, LP-184 shows ˜20% brain tumor exposure relative to plasma (based on AUC); and ˜2-fold higher brain tumor exposure compared to normal brain.

TABLE 1
Pharmacokinetic parameters of LP-184 bioavailability in brain

| | | | Pharmacokinetic Data (Mean ± SEM) | | | | Brain |
|---|---|---|---|---|---|---|---|
| Measured | | | Cₘₐₓ | | AUC₀₋ₜ | Half- | Tissue/ |
| Analyte | | Dosing | (ng/g | Tₘₐₓ | (ng · h/g Or | Life | Plasma |
| & Dose | Matrix | Route | or ng/ml) | (h) | ng · h/mL) | (h) | Ratio |
| LP-184 | Plasma | IV | 3438 ± 125 | 0.0833 | 1592 ± 48.7 | 0.236 | — |
| 4 mg/kg | Peritumoral | | 279 ± 20.2 | 0.25 | 165 ± 11.6 | 0.383 | 0.103 |
| | Normal | | | | | | |
| | Brain | | | | | | |
| | Contralateral | | 223 ± 50.6 | 0.25 | 123 ± 15.9 | 0.444 | 0.077 |
| | Normal | | | | | | |
| | Brain | | | | | | |
| | Brain Tumor | | 773 ± 58.6 | 0.0833 | 319 ± 36.4 | 0.281 | 0.200 |

Moreover, LP-184 exhibits slower clearance in mice with tumors compared to non-tumored mice (2389 ml/h/kg vs 5284 ml/h/kg) that could lead to higher plasma exposures.

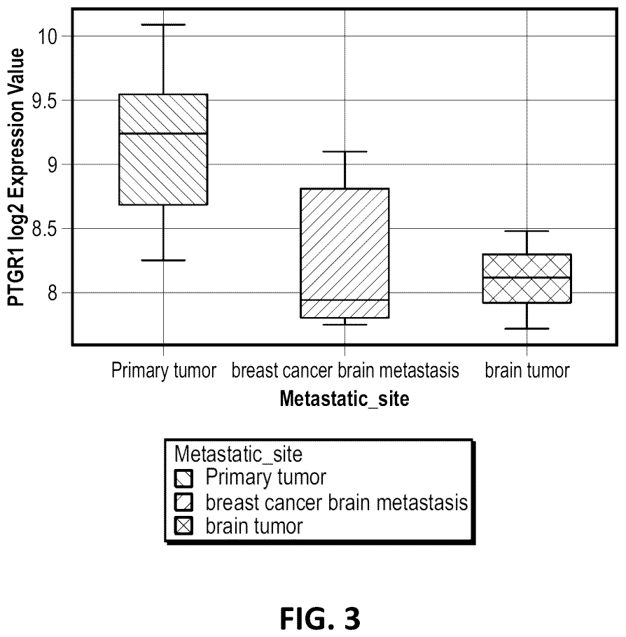

FIG. 3 shows that analysis of GSE100534 demonstrated that PTGR1 levels in brain metastatic tissue (from primary breast tumors) are above the threshold for LP-184 sensitivity.

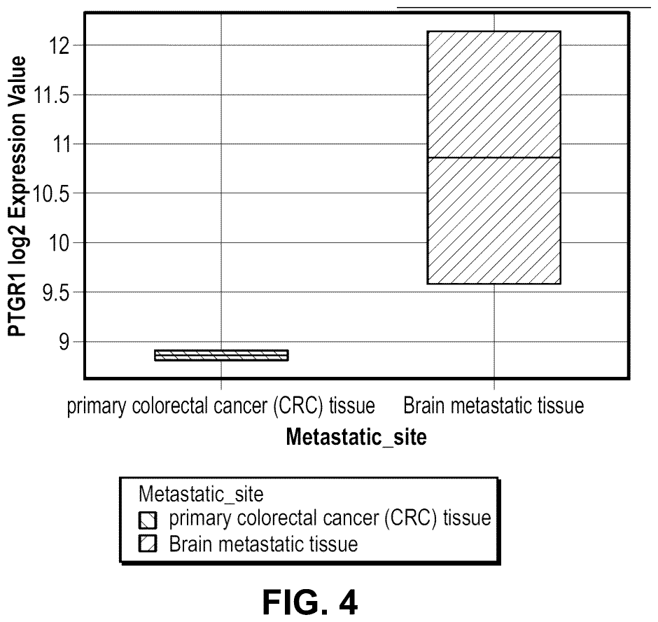

FIG. 4 shows that analysis of GSE132226 demonstrated that PTGR1 levels in brain metastatic tissue (from primary colorectal tumors) are above the threshold for LP-184 sensitivity.

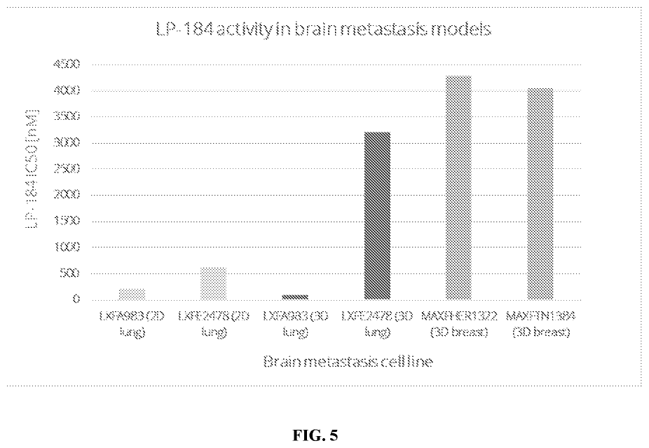

FIG. 5 shows that LP-184 retains potency in 3D models of patient-derived xenograft (PDX) derived brain metastases. Various brain metastasis models of primary lung and breast cancers showed dose-dependent sensitivity to LP-184 from low nanomolar to micromolar range, depending on the testing conditions. LP-184 IC50s over a 72-hour treatment period were found to be in the range of 88 nM to 4.283 μM.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

### Sequences:

```
Prostaglandin Reductase 1 (PTGR1) isoform 1 [Homo sapiens]:
SEQ ID NO: 1
    1 mvrtktwtlk khfvgyptns dfelktaelp plkngevlle alfltvdpym rvaakrlkeg
  61 dtmmgqqvak vvesknvalp kgtivlaspg wtthsisdgk dleklltewp dtiplslalg
121 tvgmpgltay fglleicgvk ggetvmvnaa agavgsvvgq iaklkgckvv gavgsdekva
181 ylqklgfdvv fnyktvesle etlkkaspdg ydcyfdnvgg efsntvigqm kkfgriaicg
241 aistynrtgp lppgpppeiv iyqelrmeaf vvyrwqgdar qkalkdllkw vlegkiqyke
301 yiiegfenmp aafmgmlkgd nlgktivka
```

| Prostaglandin Reductase 1 (PTGR1) isoform 1 [Homo sapiens] transcript | |
|---|---|
| variant 1, mRNA: | |
| SEQ ID NO: 2 | |
| 1 | gcagtcccag ccgccttccc caggcagtgg aaccttcggg ctcctgagct tcaggatggt |
| 61 | tcgtactaag acatggaccc tgaagaagca ctttgttggc tatcctacta atagtgactt |
| 121 | tgagttgaag acagctgagc tcccaccctt aaaaaatgga gaggtcctgc ttgaagcttt |
| 181 | gttcctcacc gtggatccct acatgagagt ggcagccaaa agattgaagg aaggtgatac |
| 241 | aatgatgggg cagcaagtgg ccaaagttgt ggaaagtaaa aatgtagccc taccaaaagg |
| 301 | aactattgta ctggcttctc caggctggac aacgcactcc atttctgatg ggaaagatct |
| 361 | ggaaaagctg ctgacagagt ggccagacac aataccactg tctttggctc tggggacagt |
| 421 | tggcatgcca ggcctgactg cctactttgg cctacttgaa atctgtggtg tgaagggtgg |
| 481 | agaaacagtg atggttaatg cagcagctgg agctgtgggc tcagtcgtgg ggcagattgc |
| 541 | aaagctcaag ggctgcaaag ttgttggagc agtagggtct gatgaaaagg ttgcctacct |
| 601 | tcaaaagctt ggatttgatg tcgtctttaa ctacaagacg gtagagtctt tggaagaaac |
| 661 | cttgaagaaa gcgtctcctg atggttatga ttgttatttt gataatgtag gtggagagtt |
| 721 | ttcaaacact gttatcggcc agatgaagaa atttggaagg attgccatat gtggagccat |
| 781 | ctctacatat aacagaaccg gcccacttcc cccaggccca cccccagaga ttgttatcta |
| 841 | tcaggagctt cgcatggaag cttttgtcgt ctaccgctgg caaggagatg cccgccaaaa |
| 901 | agctctgaag gacttgctga aatgggtctt agagggtaaa atccagtaca aggaatatat |
| 961 | cattgaagga tttgaaaaca tgccagctgc atttatggga atgctgaaag gagataattt |
| 1021 | ggggaagaca atagtgaaag catgaaaaag aggacacatg gaatctggag gccatttaga |
| 1081 | tgattagtta atttgttttt caccatttag caaaaatgta tactacctta aatgtcttaa |
| 1141 | gaaatagtac tcataatgag tttgagctac ttaataaaat acatttaagt ggtatgtaat |
| 1201 | tagtgatgga ggatggaagt ttcaaagtca acaacaacca gtcacctcac tgttcactac |
| 1261 | agcttcttct gagttgtggt agaaaataat ggctttgaag tgtgaaaatt ttccaaacac |
| 1321 | tgtatgtttt cattcataag tgggagctga acaatgagaa cacatggaca tagggagggg |
| 1381 | aacaacacac gctggggcct gtggaagccg gggtgaggga gggagagcat caggataaat |
| 1441 | agctaatgcc ttcgggggct aatacctagg tgatgggttg acaggtgaag caaaccatca |
| 1501 | tggcacacgt ttacctatgt aacaaacctg cacattccac acaattatcc cggaactgaa |
| 1561 | aataaaataa aattaaatta aaaaaaaaag ttagccggg |

```
Prostaglandin Reductase 1 (PTGR1) isoform 2 [Homo sapiens]:
SEQ ID NO: 3
    1 mvrtktwtlk khfvgyptns dfelktaelp plkngevlle alfltvdpym rvaakrlkeg
  61 dtmmgqqvak vvesknvalp kgtivlaspg wtthsisdgk dleklltewp dtiplslalg
121 tvgmpgltay fglleicgvk ggetvmvnaa agavgsvvgq iaklkgckvv gavgsdekva
181 ylqklgfdvv fnyktvesle etlkkaspdg ydcyfdnvgg efsntvigqm kkfgriaicg
241 aistynrtgp lppgpppeiv iyqelrmeaf vvyrwqgdar qkalkdllkw vleikrenee
301 d
```

```
Prostaglandin Reductase 1 (PTGR1) isoform 2 [Homo sapiens] transcript
variant 3, mRNA:
SEQ ID NO: 4
    1 gcagtcccag ccgccttccc caggcagtgg aaccttcggg ctcctgagct tcaggatggt
  61 tcgtactaag acatggaccc tgaagaagca ctttgttggc tatcctacta atagtgactt
121 tgagttgaag acagctgagc tcccaccctt aaaaaatgga gaggtcctgc ttgaagcttt
181 gttcctcacc gtggatccct acatgagagt ggcagccaaa agattgaagg aaggtgatac
241 aatgatgggg cagcaagtgg ccaaagttgt ggaaagtaaa aatgtagccc taccaaaagg
301 aactattgta ctggcttctc caggctggac aacgcactcc atttctgatg ggaaagatct
361 ggaaaagctg ctgacagagt ggccagacac aataccactg tctttggctc tggggacagt
421 tggcatgcca ggcctgactg cctactttgg cctacttgaa atctgtggtg tgaagggtgg
481 agaaacagtg atggttaatg cagcagctgg agctgtgggc tcagtcgtgg ggcagattgc
541 aaagctcaag ggctgcaaag ttgttggagc agtagggtct gatgaaaagg ttgcctacct
601 tcaaaagctt ggatttgatg tcgtctttaa ctacaagacg gtagagtctt tggaagaaac
661 cttgaagaaa gcgtctcctg atggttatga ttgttatttt gataatgtag gtggagagtt
721 ttcaaacact gttatcggcc agatgaagaa atttggaagg attgccatat gtggagccat
781 ctctacatat aacagaaccg gcccacttcc cccaggccca cccccagaga ttgttatcta
841 tcaggagctt cgcatggaag cttttgtgt ctaccgctgg caaggagatg cccgccaaaa
901 agctctgaag gacttgctga aatgggtctt agagatcaaa agagaaaatg aagaagattg
961 aagcttcaaa gcagaaaaat gaagggaata tgtatcattc accattacct ta
```

1. A method of treating, decreasing, or suppressing a cancer that has metastasized to the brain in a subject, comprising (a) diagnosing the subject as having brain metastasis; and (b) administering to the subject a therapeutically effective amount of HydroxyUreaMethyl Acylfulvene, wherein the HydroxyUreaMethyl Acylfulvene has a negative optical activity.

2. The method according to claim 1, wherein the Hydroxy UreaMethyl Acylfulvene is administered as a monotherapy.

3. The method according to claim 1, wherein the HydroxyUreaMethyl Acylfulvene is administered as a combination therapy.

4. The method according to claim 3, wherein the combination therapy comprises administration of HydroxyUreaMethyl Acylfulvene and at least one therapeutic agent selected from the group consisting of temozolomide, bevacizumab, everolimus, carmustine, lomustine, procarbazine, vincristine, irinotecan, cisplatin, carboplatin, paclitaxel, methotrexate, etoposide, vinblastine, bleomycin, actinomycin, cyclophosphamide, and ifosfamide.

5. The method according to claim 1, further comprising subjecting the subject to a radiation therapy.

6. The method according to claim 6, wherein the radiation therapy is selected from the group consisting of whole-brain irradiation, fractionated radiotherapy, radiosurgery, and a combination thereof.

7. The method of claim 1, further comprising subjecting the subject to radiation therapy before, after, or during treatment with HydroxyUreaMethyl Acylfulvene

8. The method according to claim 1, wherein administering an effective amount of HydroxyUreaMethyl Acylfulvene to a subject in need thereof results in an inhibition of brain metastasis in the subject by more than 10%, 20%, 30%, 40%, 50%, 60%, 70%, 80%, 90%, or 100% relative to brain metastasis not exposed to the treatment.

9. The method according to claim 1, wherein the cancer is associated with an increased expression of prostaglandin reductase 1 (PTGR1) enzyme.

10. The method according to claim 1, further comprising: (a) determining expression of PTGR 1 gene in a sample obtained from the subject; (b) selecting the subject having an increased expression level of PTGR1 gene in step a; and (c) administering to the subject selected in step b an effective amount of HydroxyUreaMethyl Acylfulvene, thereby treating, supressing or decreasing brain metastasis in the subject.

11. The method according to claim 1, wherein the PTGR1 gene comprises the nucleic acid sequence as set forth in SEQ ID NO:2 or SEQ ID NO:4.

12. The method according to claim 1, comprising: (a) determining expression of PTGR 1 protein in a sample obtained from the subject; (b) selecting the subject having an increased expression level of PTGR1 protein in step a; and (c) administering to the subject selected in step b an effective amount of HydroxyUreaMethyl Acylfulvene, thereby treating or decreasing brain metastasis in the subject.

13. The method according to claim 1, wherein the PTGR 1 protein comprises the amino acid sequence as set forth in SEQ ID NO: 1 or SEQ ID NO:3.

14. A method of inhibiting or decreasing brain metastasis or CNS metastasis in a subject having a cancer, comprising:
(a) determining or diagnosing the subject as having brain metastasis or CNS metastasis;
(b) measuring the level of expression of PTGR1 gene or the protein for which it encodes in a biological sample obtained from the subject,
(c) comparing the level of expression of PTGR1 gene or the protein for which it encodes in said sample against a standard of expression of PTGR1 gene or the protein for which it encodes, wherein the level of expression of PTGR1 gene or the protein for which it encodes is increased or high; and
(d) administering a therapeutically effective amount of Hydroxy UreaMethyl Acylfulvene thereby inhibiting or decreasing the brain metastasis.

15. A method of inhibiting or decreasing brain metastasis in a subject having a cancer, comprising:
(a) measuring the level of expression of PTGR1 gene or the protein for which it encodes in a biological sample obtained from the subject,
(b) comparing the level of expression of PTGR1 gene or the protein for which it encodes in said sample against a standard of expression of PTGR1 gene or the protein for which it encodes, and
(c) where the level of expression of PTGR1 gene or the protein for which it encodes is at or above a threshold for HydroxyUreaMethyl Acylfulvene sensitivity, administering a therapeutically effective amount of HydroxyUreaMethyl Acylfulvene thereby inhibiting or decreasing the brain metastasis.

16. The method according to claim 1 or 15, wherein determining the HydroxyUreaMethyl Acylfulvene sensitivity comprises using a 3D model of PDX-derived brain metastasis.

17. A kit, for use in determining sensitivity of a specimen to HydroxyUreaMethyl Acylfulvene according to the method of any one of claim 1-16, wherein the kit comprises one or more reagents, standards, and instructions for use thereof, wherein the standards comprise expression or transcription of PTGR1, providing a threshold level, or a target level for screening sensitivity of the specimen to the HydroxyUreaMethyl Acylfulvene.
